(19) United States
(12) Patent Application Publication
(10) Pub. No.: US 2007/0299271 A1
(43) Pub. Date: Dec. 27, 2007

(54) Process for separating saturated and unsaturated fatty acids for producing cold-tolorant biodiesel fuel from soy oil

(76) Inventors: Udaya Nayanskantha Wanasundara, Saskatoon (CA); Roger Peterson, Clarks Grove, MN (US)

Correspondence Address: SHERRILL LAW OFFICES, 4756 BANNING AVE, SUITE 212, WHITE BEAR LAKE, MN, 55110-3205, US

(21) Appl. No.: 11/475,596

(22) Filed: Jun. 27, 2006

(51) Int. Cl. C07C 51/00

(52) U.S. Cl. 554/154

(57) ABSTRACT

A process for separating a blend of saturated and unsaturated fatty acids or saturated and unsaturated fatty acid alkyl esters into a first fraction enriched with the saturated fatty acids or saturated fatty acid alkyl esters and a second fraction enriched with unsaturated fatty acids or unsaturated fatty acid alkyl esters. When separating fatty acids, the process includes the steps of (a) saponifying a blend of long chain saturated and unsaturated fatty acids to form free fatty acids, (b) complexing the free fatty acids with urea, and (c) separating the urea complexed free fatty acids into a first fraction enriched with saturated free fatty acids and a second fraction enriched with unsaturated free fatty acids. When separating fatty acid alkyl esters, the process includes the steps of (i) complexing the fatty acid alkyl esters with urea, and (ii) separating the urea complexed fatty acid alkyl esters into a first fraction enriched with saturated fatty acid alkyl esters and a second fraction enriched with unsaturated fatty acid alkyl esters.

### BACKGROUND

Biodiesel is an alternative fuel for diesel engines that is produced by the transesterification of oils and fats from plant and animal sources, commonly rapeseed oil and soy oil. The transesterification process combines the oils and/or fats with an alcohol to produce organic esters, with glycerin produced as a byproduct of the reaction. These organic esters as known as biodiesel. Biodiesel is a nontoxic, biodegradable and renewable alternative to traditional diesel fuel produced from crude oil. Biodiesel also tends to produce lower emissions of carbon monoxide in most engines.

The accelerating worldwide demand for energy is driving an ever expanding search for alternative energy sources, including the search for improved processes and methods for producing biodiesel.

One of the drawbacks associated with the use of traditional biodiesel fuel in the gelling of biodiesel at cold temperatures such as those commonly encountered in the northern portion of the United States and Canada during the winter.

Accordingly, a need also exists for a cold-tolerant biodiesel fuel.

### SUMMARY OF THE INVENTION

A first aspect of the invention is a process for separating a blend of saturated and unsaturated fatty acids, such as found in soy oil, into a first fraction enriched with saturated fatty acids and depleted of unsaturated fatty acids and a second fraction enriched with unsaturated fatty acids and depleted of saturated fatty acids. The process includes the steps of (a) saponifying a blend of long chain saturated and unsaturated fatty acids to form free fatty acids, (b) complexing the free fatty acids with urea, and (c) separating the urea complexed free fatty acids into a first fraction and a second fraction wherein the first fraction is enriched with saturated free fatty acids and depleted of unsaturated free fatty acids and the second fraction is enriched with unsaturated free fatty acids and depleted of saturated free fatty acids.

The unsaturated free fatty acids in the second fraction may be transesterified to form fatty acid alkyl esters suitable for use as a cold-tolerant biodiesel fuel.

A second aspect of the invention is a process for separating a blend of saturated and unsaturated fatty acid alkyl esters, such as found in transesterified soy oil, into a first fraction enriched with saturated fatty acid alkyl esters and depleted of unsaturated fatty acid alkyl esters and a second fraction enriched with unsaturated fatty acid alkyl esters and depleted of saturated fatty acid alkyl esters. The process includes the steps of (a) complexing the fatty acid alkyl esters with urea, and (b) separating the urea complexed fatty acid alkyl esters into a first fraction and a second fraction wherein the first fraction is enriched with saturated fatty acid alkyl esters and depleted of unsaturated fatty acid alkyl esters and the second fraction is enriched with unsaturated fatty acid alkyl esters and depleted of saturated fatty acid alkyl esters.

### DETAILED DESCRIPTION OF THE INVENTION INCLUDING A BEST MODE

### Definitions

As utilized herein, including the claims, the term “depleted” means lessened in quantity or content.

As utilized herein, including the claims, the term “enriched” means increased in quantity or content.

As utilized herein, including the claims, the term “selective” means to take by preference so as to increase the percentage of the selected object(s), item(s) or thing(s) in the selected portion.

### The Process

The first aspect of the invention is a process for separating a blend of saturated and unsaturated fatty acids into a first fraction enriched with saturated fatty acids and depleted of unsaturated fatty acids and a second fraction enriched with unsaturated fatty acids and depleted of saturated fatty acids. The blend of saturated and unsaturated fatty acids may be obtained from various known plant and animal sources including rapeseed oil and soy oil.

Separation of the saturated fatty acids from the unsaturated fatty acids can be achieved by complexing a blend of the fatty acids with urea. Urea has been found to selectively complex saturated fatty acids relative to unsaturated fatty acids, creating a saturated fatty acid enriched solids fraction and an unsaturated fatty acid enriched liquid fraction. The solvent of choice for use in this step of the process is a C₁₋₃ alcohol with or without water at a weight ratio of at least about 2:1 solvent to urea, preferably about 3:1 to 10:1, most preferably about 4:1 to 5:1. A weight ratio of less than about 2:1 tends to result in incomplete complexation of the unsaturated fatty acids while a weight ratio in excess of about 10:1 increases processing cost without a concomitant increase in yield or processability. Urea should be employed at a weight ratio of at least 1:1 urea to fatty acid, preferably at a weight ratio of about 2:1 to 5:1, most preferably about 3:1. A weight ratio of less than about 1:1 tends to result in incomplete complexation of the fatty acids while a weight ratio in excess of about 5:1 increases processing cost without a concomitant increase in yield or separation efficiency.

Separation of the saturated fatty acid enriched solids fraction and unsaturated fatty acid enriched liquid fraction can be achieved by any of the well-known solid-liquid separation techniques. Suitable processes and systems include specifically, but not exclusively, decantation, countercurrent decantation, gravity sedimentation, filtration, expression, centrifugation and combinations thereof. The preferred method is filtration.

The unsaturated fatty acid enriched liquid fraction can be washed with water acidified to a pH of about 3-4, followed by separation of the resultant aqueous and oil phases, to remove impurities.

When oil is used as the starting source of blended saturated and unsaturated fayy acids, the oil is preferably saponified prior to treatment with urea. Saponification of the oil frees the fatty acids contained in the oil so as to increase availability of the fatty acids for complexing with urea. Saponification can be achieved by adding a suitable solvent to the oil, such as a short chain alcohol with or without water, and a hydrolyzing reagent such as lye or other caustic. The solvent of choice for use in this step of the process is a C₁₋₃ alcohol with or without water at a weight ratio of at least about 2:1 solvent to oil, preferably about 2:1 to 10:1, most preferably about 3:1 to 4:1. A weight ratio of less than about 2:1 tends to result in incomplete saponification of the unsaturated fatty acids while a weight ratio in excess of about 10:1 increases processing cost without a concomitant increase in yield or processability. The caustic, such as NaOH, should be employed at a weight ratio of at least 1:20 caustic to oil, preferably at a weight ratio of about 2:10 to 5:10, most preferably about 3:10. A weight ratio of less than about 1:20 tends to result in incomplete saponificaiton of the unsaturated fatty acids while a weight ratio in excess of about 1:20 increases processing cost without a concomitant increase in saponificaiton yield or efficiency.

The saponified oil can then be separated by any suitable separation technique into a glycerine enriched fraction, a free fatty acid enriched fraction and a waste fraction containing the solvent and other constituents from the oil. Suitable separation techniques include any of the well-known techniques for separating such organic liquid-liquid systems including centrifugation, decantation and distillation. A preferred technique includes acidification of the mixture with a suitable acid, such as sulfuric acid or citric acid, followed by centrifugation. The glycerine enriched fraction can be redirected for further processing into commercially saleable glycerine. The waste fraction can be redirected for solvent recovery and recycle.

The unsaturated fatty acids in the unsaturated fatty acid enriched liquid fraction can then be transesterified in accordance with any of the well known techniques for transesterification of fatty acids to produce biodiesel.

The resultant unsaturated fatty acid alkyl esters (i.e., methyl and/or ethyl esters) can be washed with water acidified to a pH of about 3-4, followed by separation of the resultant aqueous and oil phases, to remove impurities.

The second aspect of the invention is a process for separating a blend of saturated and unsaturated fatty acid alkyl esters into a first fraction enriched with saturated fatty acid alkyl esters and depleted of unsaturated fatty acid alkyl esters and a second fraction enriched with unsaturated fatty acid alkyl esters and depleted of saturated fatty acid alkyl esters. The blend of saturated and unsaturated fatty acid alkyl esters may be obtained by traditional transesterification of various known plant and animal sources including rapeseed oil and soy oil. In other words, the second aspect of the invention starts with traditional biodiesel.

As with the first aspect of the invention, separation of the saturated fatty acid alkyl esters from the unsaturated fatty acid alkyl esters can be achieved by complexing a blend of the fatty acid alkyl esters (i.e., methyl and/or ethyl esters) with urea. As with the first aspect of the invention, the solvent of choice for use in this step of the process is a C₁₋₃ alcohol with or without water at a weight ratio of at least about 2:1 solvent to urea, preferably about 3:1 to 10:1, most preferably about 4:1 to 5:1. A weight ratio of less than about 2:1 tends to result in incomplete complexation of the unsaturated fatty acid alkyl esters while a weight ratio in excess of about 10:1 increases processing cost without a concomitant increase in yield or processability. Urea should be employed at a weight ratio of at least 1:1 urea to fatty acid ester, preferably at a weight ratio of about 2:1 to 5:1, most preferably about 3:1. A weight ratio of less than about 1:1 tends to result in incomplete complexation of the fatty acid esters while a weight ratio in excess of about 5:1 increases processing cost without a concomitant increase in yield or separation efficiency.

Again, as with the first aspect of the invention, the saturated fatty acid alkyl ester enriched solids fraction and unsaturated fatty acid alkyl ester enriched liquid fraction can be achieved by any of the well-known solid-liquid separation techniques. Suitable processes and systems include specifically, but not exclusively, decantation, countercurrent decantation, gravity sedimentation, filtration, expression, centrifugation and combinations thereof. The preferred method is filtration.

The unsaturated fatty acid alkyl ester enriched liquid fraction can be washed with water acidified to a pH of about 3-4, followed by separation of the resultant aqueous and oil phases, to remove impurities.

### EXAMPLES

### Glossary

| ACRONYM | DESCRIPTION |
|---|---|
| FFA | Free Fatty Acid |
| SFFA | Saturated Free Fatty Acid |
| UFFA | Unsaturated Free Fatty Acid |
| PUFFA | Polyunsaturated Free Fatty Acid |
| FAME | Fatty Acid Methyl Ester |
| SFAME | Saturated Fatty Acid Methyl Ester |
| UFAME | Unsaturated Fatty Acid Methyl Ester |

### Example 1

### (Prophetic)

### Separation of Soy Oil

Into a reaction vessel is introduced 1000 grams of refined soy oil, 180 grams of powdered sodium hydoxide, 440 grams of water and 2100 grams ethanol (95% by weight) to form a first admixture. The first admixture is agitated for 1.5 hours at 60° to 65° C. after which the first admixture cooled to approximately 25° C., an additional 2,000 grams of water is introduced, and sufficient acid is added to adjust the pH of the first admixture to 2-3.

The cooled first admixture is separated by a three way centrifuge into a glycerine enriched fraction, a FFA enriched fraction and a waste fraction containing ethanol (95% by weight), water, acid and the sodium salt formed as the reaction product of the sodium hydroxide and acid. The glycerine enriched fraction is redirected for further processing into commercially saleable glycerine. The waste fraction is redirected for recovery and recycle.

The FFA enriched fraction is introduced into a second reaction vessel. A sufficient amount of urea, dissolved in ethanol (95% by weight) is added to the FFA enriched fraction on the second reaction vessel at a urea to FFA ratio of 1:1 (w/w/) and a urea to solvent concentration of 40% (w/v) to form a second admixture. The second admixture is blended together and then allowed to stand for four hours at 20° C. The second admixture is then filtered to produce an UFFA enriched filtrate and a SFFA enriched retentate containing SFFA and urea.

The SFFA enriched retentate is introduced into a first mixing vessel along with 40,000 grams of water to form a third admixture. The third admixture is blended together at 60° C. until the urea is fully dissolved. The third admixture is then separated by centrifugation into an aqueous phase containing the urea, and an oil phase containing the SFFA. The aqueous phase is redirected for recovery and recycle.

The oil phase is washed with acidified water and the washed SFFAs separated by chromatography to produce myristic acid, palmitic acid, stearic acid, oleic acid and additional recovered UFFAs.

The UFFA enriched filtrate is washed twice with acidified water (pH 3-4) and dried.

The dried UFFA enriched filtrate and the additional recovered UFFAs are introduced into a third reaction vessel along with 400 grams of ethanol (95% by weight) and 50 grams of sulfuric acid to form a fourth admixture. The fourth admixture is agitated at room temperature for 1-2 hours until transesterification of the UFFAs is complete. Crude FAMEs are recovered from the fourth admixture and washed with a 10 wt % aqueous solution of sodium chloride at 50° to 60° C. The washed FAMEs are separated from the wash water by centrifugation and stored.

### Example 2

### (Prophetic)

### Separation of Transesterified Soy Oil

Into a reaction vessel is introduced 140 grams of fatty acid methyl esters obtained by transesterification of soy oil, and 390 grams of urea dissolved in 200 grams of ethanol (95% by weight) to form a first admixture. The first admixture is blended together and then allowed to stand for four hours at 20° C. The first admixture is then filtered to produce an UFAME enriched filtrate and a SFAME enriched solids containing SFAME and urea.

The SFAME enriched solids is introduced into a first mixing vessel along with 450 grams of water to form a second admixture. The second admixture is blended together at 60° C. until the urea is fully dissolved. The second admixture is then separated by centrifugation into an aqueous phase containing the urea, and an oil phase containing the SFAME. The aqueous phase is redirected for recovery and recycle.

The oil phase is washed with acidified water and the washed SFAMEs separated by chromatography to produce purified methyl esters of myristic acid, palmitic acid, stearic acid, oleic acid and additional recovered UFAMEs.

The UFAME enriched filtrate is washed twice with acidified water (pH 3-4) and stored.

1. A process, comprising:
(a) saponifying soy oil having a blend of long chain saturated and unsaturated fatty acids to form free fatty acids,
(b) complexing the free fatty acids with urea, and
(c) separating the urea complexed free fatty acids into a first fraction and a second fraction wherein the first fraction is enriched with saturated free fatty acids and depleted of unsaturated free fatty acids and the second fraction is enriched with unsaturated free fatty acids and depleted of saturated free fatty acids.

2. The process of claim 1 wherein the process further comprises transesterification of the second fraction to form long chain unsaturated fatty acid alkyl esters suitable for use as a biodiesel fuel.

3. The process of claim 1 wherein the soy oil is refined soy oil.

4. The process of claim 3 wherein the soy oil is saponified by admixing the soy oil with a C₁₋₃ alcohol or a combination of a C₁₋₃ alcohol and water at an elevated temperature in the presence of an alkali catalyst to form a first admixture.

5. The process of claim 4 wherein the C₁₋₃ alcohol is ethanol and a majority of the ethanol is recycled ethanol recovered from the process.

6. The process of claim 5 further comprising the steps of (i) adjusting the pH of the first admixture below 6 by the addition of an acid after saponification of the soy oil, and (ii) recovering a free fatty acid enriched fraction and a glycerine enriched fraction from the pH adjusted first admixture wherein the recovered free fatty acid enriched fraction is the source of free fatty acids complexed with urea in step (b).

7. The process of claim 1 wherein the free fatty acids are complexed with urea by admixing the free fatty acids with urea dissolved in a C₁₋₃ alcohol or a combination of a C₁₋₃ alcohol and water to form a second admixture.

8. The process of claim 1 wherein complexation of the free fatty acids with urea forms a saturated free fatty acid enriched solids fraction and an unsaturated free fatty acid enriched liquids fraction.

9. The process of claim 8 wherein the step of separating the urea complexed free fatty acids into a first fraction and a second fraction comprises the step of separating the solids fraction and the liquids fraction.

10. The process of claim 9 wherein the solids fraction and the liquids fraction are separated by filtration.

11. The process of claim 1 further comprising the step of separating residual unsaturated free fatty acids in the first fraction from the saturated free fatty acids in the first fraction to obtain additional unsaturated free fatty acids.

12. The process of claim 11 further comprising the step of transesterification of the additional unsaturated free fatty acids to form long chain unsaturated fatty acid alkyl esters suitable for use as a biodiesel fuel.

13. The process of claim 11 wherein the residual unsaturated free fatty acids are separated from the saturated free fatty acids in the first fraction by chromatography.

14. The process of claim 2 wherein the alkyl esters are ethyl esters.

15. A process, comprising:
(a) complexing soy oil containing a blend of long chain saturated and unsaturated fatty acid alkyl esters with urea, and
(b) separating the urea complexed fatty acid alkyl esters into a first fraction and a second fraction wherein the first fraction is enriched with saturated fatty acid alkyl esters and depleted of unsaturated fatty acid alkyl esters and the second fraction is enriched with unsaturated fatty acid alkyl esters and depleted of saturated fatty acid alkyl esters.

16. The process of claim 15 wherein the blend of long chain saturated and unsaturated fatty acids is transesterified soy oil.

17. The process of claim 15 wherein the fatty acid alkyl esters are complexed with urea by admixing the fatty acid alkyl esters with urea dissolved in a C₁₋₃ alcohol or a combination of a C₁₋₃ alcohol and water to form a first admixture.

18. The process of claim 15 wherein complexation of the fatty acid alkyl esters with urea forms a saturated fatty acid allcyl ester solids fraction and an unsaturated fatty acid alkyl ester enriched liquids fraction.

19. The process of claim 18 wherein the step of separating the urea complexed fatty acid alkyl esters into a first fraction and a second fraction comprises the step of separating the solids fraction and the liquids fraction.

20. The process of claim 19 wherein the solids fraction and the liquids fraction are separated by filtration.

21. The process of claim 15 further comprising the step of separating residual unsaturated free fatty acid alkyl esters in the first fraction from the saturated free fatty acid alkyl esters in the first fraction to obtain additional unsaturated free fatty acid alkyl esters.

22. The process of claim 22 wherein the residual unsaturated free fatty acid alkyl esters are separated from the saturated free fatty acid alkyl esters in the first fraction by chromatography.

23. The process of claim 15 wherein the fatty acid alkyl esters are fatty acid ethyl esters.
